(19) United States
(12) Patent Application Publication
(10) Pub. No.: US 2018/0125092 A1
(43) Pub. Date: May 10, 2018

(54) METHOD FOR PREPARING FEEDSTUFFS COMPRISING BUTYRIC ACID AND/OR BUTYRATE

(71) Applicant: Green Cellulosity Corporation, Hsinchu City (TW)

(72) Inventors: Chiang-Hsiung TONG, Hsinchu City (TW); Shih-Chan TSENG, Hsinchu City (TW); Chun-Han CHEN, Hsinchu City (TW); Wan-Shan CHIEN, Hsinchu City (TW); Jheng-Jin LUO, Hsinchu City (TW); Ruey-Fu SHIH, Hsinchu City (TW)

(21) Appl. No.: 15/790,982

(22) Filed: Oct. 23, 2017

Related U.S. Application Data
(60) Provisional application No. 62/419,102, filed on Nov. 8, 2016.

(30) Foreign Application Priority Data
Aug. 17, 2017 (TW) 106127954

(51) Int. Cl. A23K 10/12; A23K 10/37; C12P 7/52; C12P 7/62; C12N 1/20; C08B 30/04

(52) U.S. Cl. CPC A23K 10/12; A23K 10/37; C08B 30/044; C12P 7/62; C12N 1/20; C12P 7/52

(57) ABSTRACT

A method for preparing a feedstuff comprising butyric acid and/or butyrate, comprising: adding a microorganism into a light corn steepwater to provide a mixture, wherein the microorganism comprises a first strain and the first strain is able to metabolize saccharides and/or organic compounds in a fermentation to produce butyric acid; keeping the mixture under an anaerobic atmosphere to conduct the fermentation to provide a fermentation broth; and optionally condensing the fermentation broth. Optionally, the microorganism further comprises a second strain, wherein the second strain is able to fix a carbon oxide.

### CROSS REFERENCE TO RELATED APPLICATIONS

This application claims priority to U.S. Provisional Application Ser. No. 62/419,102 filed on Nov. 8, 2016, in the United States Patent and Trademark Office, and to Taiwan Patent Application No. 106127954 filed on Aug. 18, 2017, in the Taiwan Intellectual Property Office; the disclosures of which are incorporated herein in their entirety by reference.

### FIELD OF THE INVENTION

The present invention relates to the uses of microorganisms in preparing a feedstuff, especially the uses of microorganisms in preparing a feedstuff comprising butyric acid and/or butyrate without externally adding butyric acid and butyrate. Specifically, the method of the present invention uses microorganisms and light corn steepwater to provide a fermentation broth comprising butyric acid and/or butyrate without externally adding butyric acid and butyrate. The fermentation broth can be used for preparing animal feeds.

### BACKGROUND OF THE INVENTION

Corn kernels are commonly used in the manufacture of animal feeds and their major ingredients are starch, protein, fat and corn hull fiber. The processing flow of manufacturing feed from corn can be generally divided into two types, i.e., wet-methods and dry-methods. The wet-method refers to a method for providing a high purity starch product and comprising the steps of steeping corn kernels into warm water and shattering the steeped corn kernels to separate and obtain therefrom germ, fiber and protein. FIG. 1 shows the processing flow of a typical wet-method for producing corn starch.

As shown in FIG. 1, in the processing flow of a typical wet-method for producing corn starch, corn kernel A is subjected to a cleansing processing 100 prior to being steeped into warm water. In the cleansing processing 100, corn kernel A is washed with water B to remove impurities and dust. Thereafter, corn kernel A is subject to steeping processing 200 by steeping corn kernel A into warm water (such as warm water at a temperature of about 46-52° C.) in which sulfur dioxide C is introduced (for such as 40-80 hours) to obtain a light corn steepwater D and soften corn kernel. Steepwater D comprises crude protein, ash, carbohydrate and fat. Condensed corn extractives E (also called as “corn steep liquor” or “corn steepwater”) with an enhanced solid content (such as up to about 45 to 55 wt %) can be obtained by subjecting steepwater D to evaporation-condensation processing 210. Condensed corn extractives E can be further dried to provide a total solid corn steep powder.

As shown in FIG. 1, the softened corn kernel is subjected to breakdown processing 300 and then germ isolation processing 400 to separate the germ of corn, and the residue of germ isolation processing 400 is subjected to a fine grinding processing 500 and then fiber isolation processing 600 to separate corn hull fiber K. The residue of fiber isolation processing 600 is subjected to protein isolation processing 700 to remove protein, and the starch obtained from protein isolation processing 700 is subjected to starch wash 800 with water F, dehydration 810 and drying 820 to provide corn starch G. The corn germ obtained from germ isolation processing 400 is subjected to germ wash 410, dehydration 420 and drying 430 to provide germ H. Germ H could be further subjected to oil press processing 440 to provide crude corn oil I and corn germ meal J. The corn hull fiber K obtained from the fiber isolation processing 600 is further subjected to wash and dehydration processing 610, mixing 620, drying 630 and granulation 640 to provide corn gluten feed L that can be used for feeding animals, wherein mixing 620 is carried out with the addition of condensed corn extractives E obtained from evaporation-condensation processing 210 and optionally together with the corn germ meal J from oil press processing 440. In addition, the protein obtained from the protein isolation processing 700 is subjected to evaporation-condensation processing 710, dehydration 720 and drying 730 to provide corn gluten meal M.

In the past, antibiotics are regularly added into feeds to achieve the effects such as maintaining health of animals, promoting growth of animals and enhancing the utilization rate of feeds. However, people have been concerned with drug resistance and drug residue problems caused by the use of antibiotics, and thus, governments all over the world have taken corresponding measures to strictly control the use of antibiotics as a feed additive. Therefore, in the study of feed additives, researchers have focused on developing substitutes for antibiotics.

It has been known that the presence of butyric acid or butyrate (e.g., sodium butyrate) in animal feeds can provide many benefits for the fed animals, including defending against bacteria, inhibiting pathogens, improving cell morphology and structure of epithelial cells in the gastrointestinal tract, improving balance of intestinal microflora, promoting digestion and absorption abilities, inhibiting inflammatory reaction in the intestinal tract, and increasing immunity. Thus, the presence of butyric acid or butyrate (e.g., sodium butyrate) in animal feeds can enhance the utilization rate of feeds, increase the growth rate of fed animals and the feed conversion rate. However, according to the current processing flow (such as the processing flow of wet-method for producing corn starch as shown in FIG. 1) for the manufacture of feeds from corn, unless externally adding butyric acid and butyrate, the feed (e.g., corn gluten feed) thus provided is free of butyric acid and butyrate. In addition, the butyric acid and butyrate being added into the feeds should be of fodder-grade, but not of chemical-grade with low-price. If the low-priced butyric acid and butyrate (e.g., sodium butyrate) of chemical-grade is added into the feed, the mucous membranes of fed animals would be burned, which makes the animals decrease the intake of feeds and, for the worst situation, the intake could be zero.

Inventors of the present invention found that with the use of microorganisms in the wet-processing flow of manufacturing feed from corn, a light corn steepwater comprising butyric acid and/or butyrate that meets the requirements of fodder-grade can be directly provided in the upstreaming of a traditional processing flow without changing the processing order. The light corn steepwater comprising butyric acid and/or butyrate can be used for preparing animal feeds without externally adding butyric acid or butyrate.

### SUMMARY OF THE INVENTION

Accordingly, an objective of the present invention is to provide a method for preparing a feedstuff comprising butyric acid and/or butyrate, and the method comprises: adding a microorganism into a light corn steepwater to provide a mixture, wherein the microorganism comprises a first strain and the first strain is able to metabolize saccharides and/or organic compounds in a fermentation to produce butyric acid; keeping the mixture under an anaerobic atmosphere to conduct the fermentation to provide a fermentation broth; and optionally condensing the fermentation broth. Optionally, the microorganism further comprises a second strain and the second strain is able to fix a carbon oxide.

The detailed technology and some particular embodiments implemented for the present invention are described in the following paragraphs for people skilled in this field to well appreciate the features of the claimed invention.

### BRIEF DESCRIPTION OF THE DRAWINGS

FIG. 1 shows the processing flow of a typical wet-method for producing corn starch.

FIG. 2 shows a processing flow for producing animal feeds from corn by applying the method in accordance with the present invention.

### DESCRIPTION OF THE PREFERRED EMBODIMENTS

The following will describe some of the embodiments of the present invention in detail. However, without departing from the spirit of the present invention, the present invention may be realized in various embodiments and should not be considered to be limited to the embodiments described in the specification. In addition, unless otherwise state herein, the expressions “a,” “the” or the like recited in the specification of the present invention (especially in the claims) should include both the singular and plural forms.

The numerical ranges (e.g., 5 to 100) used in this specification should be construed as including all of the rational numbers in the ranges and ranges consisting of any rational numbers in the ranges. Therefore, the numerical ranges used in this specification should include all the possible combinations of numerical values between the lowest value and the highest value listed therein. In addition, the word “about” as used herein substantially represents values within ±20% of the stated value, preferably within ±10% and more preferably within ±5%.

The phrase “light corn steepwater” used in the specification refers to a liquid obtained by steeping corns with warm water in which sulfur dioxide is introduced, and preferably, the corns are washed with water to remove impurities and dust prior to being steeped with the sulfur dioxide-aerated warm water. Preferably, the temperature of warm water used in the steep processing is at least 40° C. (such as about 46 to 52° C.). Preferably, the steep processing is conducted for at least three days (such as for about 40 to 80 hours). The phrase “condensed corn extractives” used in the specification refers to a liquid obtained by subjecting the aforementioned steepwater to an evaporation-condensation and thus has an increased solid content (such as up to about 45 to 55 wt %). The phrase “crude protein” used in the specification is a generic term of nitrogen-containing materials, including the real proteins and nitrogen-containing substances (amides). The term “ash” used in the specification refers to the inorganic ingredients (primarily inorganic salts and oxides) remaining after foods are burned at a high temperature, which causes a series of physical and chemical changes and the organic ingredients have thus vaporized.

The term “fermentation” used in this specification refers to a process of metabolizing one or more substances by microorganism(s) under an anaerobic atmosphere to produce organic compounds. The phrase “fix a carbon oxide” used in this specification refers to a process of converting carbon oxide(s) into organic compound(s) by biochemical reaction(s). The term “microorganism” used in this specification refers to an organism that is invisible to the naked eye (such as bacteria and fungus) and includes the wild type present in nature and mutant type induced by any factors (e.g., natural factor or artificial factor).

In this specification, the term “saccharide” is also called carbohydrate and its examples include, but are not limited to, monosaccharides (e.g., glucose, fructose, galactose, mannose, arabinose, lyxose, ribose, xylose, ribulose, xylulose, allose, altrose, gulose, idose, talose, psicose, sorbose and tagatose); disaccharides (e.g., sucrose, maltose, lactose, lactulose, trehalose and cellobiose); oligosaccharides (e.g., stachyose, maltotriose, maltotetrose and maltopentaose); and polysaccharides (e.g., starch, cellulose, glycogen, cyclodextrin, arabinoxylans, guar gum, gum Arabic, chitin, gum, alginate, pectin and gellan). The term “organic compound” used in this specification, which is abbreviated as organics, refers to a carbon-containing compound (except for carbon monoxide, carbon dioxide, carbonic acid, carbonate, bicarbonate, carbide, cyanide, thiocyanide, cyanate, and metal carbide) or a generic term of hydrocarbons and the derivatives thereof.

As set forth above, in the processing flow of manufacturing animal feeds from corn according to the prior art, unless externally adding butyric acid and butyrate, the feeds (e.g., corn gluten feed) finally provided are free of butyric acid and butyrate. Therefore, butyric acid and butyrate (e.g., sodium butyrate) have to be externally added into the feeds to enhance the utilization rate of feeds, increase the growth rate of the fed animals and the feed conversion rate. Different from the prior art, inventors of the present invention found that with the use of microorganism(s) in the process of manufacturing feeds from corn, a feedstuff comprising butyric acid and/or butyrate can be directly provided, without changing the order of traditional processing flow. The feedstuffs comprising butyric acid and/or butyrate can be used for preparing corn gluten feeds comprising butyric acid and/or butyrate.

Therefore, the present invention provides a method for preparing a feedstuff comprising butyric acid and/or butyrate, comprising: adding a microorganism into a light corn steepwater to provide a mixture, wherein the microorganism comprises a first strain and the first strain is able to metabolize saccharides and/or organic compounds in a fermentation to produce butyric acid; keeping the mixture under an anaerobic atmosphere to conduct the fermentation to provide a fermentation broth; and optionally condensing the fermentation broth. Optionally, the microorganism further comprises a second strain, wherein the second strain is able to fix a carbon oxide.

In the method for preparing a feedstuff comprising butyric acid and/or butyrate in accordance with the present invention, the first strain adopted is a microorganism that is able to metabolize saccharides and/or organic compounds in a fermentation to produce butyric acid, and includes those being able to use the following pathways in a fermentation to produce butyric acid: acetyl-CoA biosynthesis pathway, butyryl-CoA biosynthesis pathway, acetone biosynthesis pathway, ethanol biosynthesis pathway, butanol biosynthesis pathway, acetate biosynthesis pathway, or acetone-butanol-ethanol (ABE), but is not limited thereto. Examples of the first strain include, but are not limited to, Clostridium sp. strain, Butyribacterium sp. strain, and Butyrivibrio sp. strain.

Examples of the Clostridium sp. strain suitable for being used as the first strain in the method of the present invention include, but are not limited to, Clostridium tyrobutyricum, Clostridium butyricum, Clostridium beijerinckii, Clostridium acetobutylicum, Clostridium argentinense, Clostridium aurantibutyricum, Clostridium botulinum, Clostridium carboxidivorans, Clostridium cellulovorans, Clostridium cf. saccharolyticum, Clostridium difficile, Clostridium kluyveri, Clostridium novyi, Clostridium paraputrificum, Clostridium pascui, Clostridium pasteurianum, Clostridium peptidivorans, Clostridium perfringens, Clostridium scatologenes, Clostridium schirmacherense, Clostridium sticklandii, Clostridium subterminale SB4, Clostridium symbiosum, Clostridium tetani, Clostridium tepidiprofundi, Clostridium tertium, Clostridium tetanomorphum and Clostridium thermopalmarium.

Examples of the Butyribacterium sp. strain suitable for being used as the first strain in the method of the present invention include, but are not limited to, Butyribacterium methylotrophicum and Butyribacterium rettgeri.

Examples of the Butyrivibrio sp. strain suitable for being used as the first strain in the method of the present invention include, but are not limited to, Butyrivibrio crossotus, Butyrivibrio fibrisolvens, Butyrivibrio hungatei and Butyrivibrio proteoclasticus.

One or more of the following strains can also be used as the first strain in the method of the present invention, but are not limited to: Anaerostipes butyraticus, Anaerostipes caccae, Anaerostipes sp., Coprococcus ART55/1, Coprococcus catus, Coprococcus comes, Coprococcus eutactus, Eubacterium biforme, Eubacterium cellulosolvens, Eubacterium dolichum, Eubacterium hadrum, Eubacterium hallii, Eubacterium L2-7, Eubacterium limosum, Eubacterium oxidoreducens, Eubacterium ramulus, Eubacterium rectale, Eubacterium saburreum, Eubacterium A2-194, Eubacterium ventriosum, Lachnospiraceae bacterium, Lachnospiraceae sp., Moryella indoligenes, Parasporobacterium paucivorans, Pseudobutyrivibrio ruminis, Pseudobutyrivibrio xylanivorans, Roseburia cecicola, Roseburia faecis, Roseburia hominis, Roseburia intestinalis, Roseburia inulinivorans, Sporobacterium olearium, Anerococcus Octavius, Peptoniphilus asaccharolyticus, Peptoniphilus, duerdenii, Peptoniphilus harei, Peptoniphilus lacrimalis, Peptoniphilus indolicus, Peptoniphilus ivorii, Peptoniphilus sp., Sedimentibacter hydroxybenzoicus, Anaerovorax odorimutans, Filifactor alocis, Eubacterium barkeri, Eubacterium infirmum, Eubacterium minutum, Eubacterium nodatum, Eubacterium sulci, Eubacterium moniliforme, Ilyobacter delafieldii, Oxobacter pfenningii, Sarcina maxima, Thermobrachium celere, Butyricicoccus pullicaecorum, Eubacterium A2-207, Gemmiger formicilis, Anaerobaculum mobile, Pelospora glutarica, Thermoanaerobacter yonseiensis, Eubacterium cylindroides, Eubacterium saphenum, Eubacterium tortuosum, Eubacterium yurii margaretiae, Peptococcus anaerobius, Peptococcus niger, Sporotomaculum hydroxybenzoicum, Acidaminococcus intestine, Acidaminococcus fermentans, Acidaminococcus sp., Megasphaera elsdenii, Megasphaera genomosp, Megasphaera micronuciformis, Halanaerobium saccharolyticum, Brachyspira intermedia, Brachyspira alvinipulli, Shuttleworthia satelles, Anaerococcus hydrogenalis, Anaerococcus lactolyticus, Anaerococcus prevotii, Anaerococcus tetradius, Anaerococcus vaginalis, Alkaliphilus metalliredigens, Alkaliphilus oremlandii, Anaerofustis stercorihominis, Pseudoramibacter alactolyticus, Anaerotruncus colihominis, Faecalibacterium cf. prausnitzii, Faecalibacterium prausnitzii, Ruminococcaceae bacterium, Subdoligranulum variabile, Thermoanaerobacterium thermosaccharolyticum, Carboxydibrachium pacificum, Carboxydothermus hydrogenoformans, Thermoanaerobacter tengcongensis, Thermoanaerobacter wiegelii, Erysipelotrichaceae bacterium, Carnobacterium sp., Desmospora sp., Acetonema longum, Thermosinus carboxydivorans, Natranaerobius thermophiles, Halanaerobium praevalens, Symbiobacterium thermophilum, Stackebrandtia nassauensis, Intrasporangium calvum, Janibacter sp., Micromonospora aurantiaca, Micromonospora sp., Salinispora arenicola, Salinispora tropica, Verrucosispora maris, Kribbella flavida, Nocardioidaceae bacterium, Nocardioides sp., Thermomonospora curvata, Haloplasma contractile, Desulfurispirillum indicum, Deferribacter desulfuricans, Rhodoferax ferrireducens and Stigmatella aurantiaca.

In addition to the above wild-type strains, the first strain used in the method of the present invention can also be a strain provided by a genetic engineering procedure, as long as the strain is able to metabolize a saccharide and/or an organic compound in a fermentation to produce butyric acid. For example, for a strain whose metabolic pathway does not include the ABE pathway or includes only part of the ABE pathway, a gene related to the ABE pathway could be inserted into the strain by genetic engineering so as to provide a strain that is able to produce butyric acid in the fermentation and thus can be used as the first strain in the method of the present invention.

In some embodiments of the method of the present invention, Clostridium tyrobutyricum is used as the first strain to metabolize a saccharide and/or an organic compound in a fermentation to produce butyric acid.

In the method for preparing a feedstuff comprising butyric acid and/or butyrate in accordance with the present invention, any microorganism that is able to fix a carbon oxide can be used as the second strain. For example, microorganisms being able to use the Wood-Ljungdahl (WL) pathway to fix a carbon oxide can be used as the second strain, but are not limited thereto.

Examples of the microorganisms that are able to use the Wood-Ljungdahl (WL) pathway to fix a carbon oxide include, but are not limited to, Clostridium coskatii, Clostridium ljungdahlii, Clostridium autoethanogenum, Clostridium ragsdalei, Terrisporobacter glycolicus, Clostridium scatologenes, Clostridium carboxidivorans, Clostridium difficile, Clostridium aceticum, Moorella thermoacetica (previously known as Clostridium thermoaceticum), Methanobacterium thermoautotrophicum, Desulfobacterium autotrophicum, Clostridium sticklandii, Clostridium thermoautotrophicum, Clostridium formicoaceticum, Clostridium magnum, Acetobacterium carbinolicum, Acetobacterium kivui, Acetobacterium woodii, Acetitomaculum ruminis, Acetoanaerobium noterae and Acetobacterium bakii.

Likewise, in addition to the above wild-type strains, the second strain used in the method of the present invention can also be a strain provided by a genetic engineering procedure. For example, for a strain whose metabolic pathway does not include the Wood-Ljungdahl (WL) pathway or includes only part of the Wood-Ljungdahl (WL) pathway, a gene related to the Wood-Ljungdahl (WL) pathway could be inserted into the strain by genetic engineering so as to provide a strain that is able to fix a carbon oxide and thus can be used as the second strain in the method of the present invention.

In the method for preparing a feedstuff comprising butyric acid and/or butyrate in accordance with the present invention, the second strain adopted is preferably at least one of the following microorganisms that is able use the Wood-Ljungdahl (WL) pathway to fix a carbon oxide: Clostridium coskatii, Clostridium ljungdahlii, Clostridium autoethanogenum, Clostridium ragsdalei, Terrisporobacter glycolicus and Clostridium scatologenes. In some embodiments of the method in accordance with the present invention, at least one of Terrisporobacter glycolicus and Clostridium ljungdahli is used as the second strain to fix a carbon oxide.

In the method for preparing a feedstuff comprising butyric acid and/or butyrate in accordance with the present invention, depending on the selected first strain (and the second strain), people having ordinary skills in the art can decide the conditions for carrying out the fermentation. For example, when Clostridium tyrobutyricum is used as the first strain and at least one of Terrisporobacter glycolicus and Clostridium ljungdahli is used as the second strain, it is preferred that the fermentation is conducted at a temperature of 32 to 42° C. (more preferably at a temperature of 34 to 40° C.) and a pH value of 4 to 8.

As known by people having ordinary skills in the art, fermentation is conducted under an anaerobic atmosphere. In the method according to the present invention, the term “anaerobic atmosphere” refers to an atmosphere that contains less than 5 ppm (part per million) of oxygen, preferably less than 0.5 ppm of oxygen, and more preferably less than 0.1 ppm of oxygen. Any suitable method can be used to provide the desired anaerobic atmosphere. For example, but not limited to, before the fermentation is performed, an inert gas (e.g., nitrogen, carbon dioxide) is introduced into the fermentation reactor to purge the reactor, and thus, provide the desired anaerobic atmosphere; alternatively, the fermentation is conducted in an anaerobic operation box, wherein a palladium catalyst is used to catalyze the reaction of the oxygen in the box and the hydrogen in the anaerobic gas mixture to produce water, and thus, provide the desired anaerobic atmosphere.

In some embodiments of the method for preparing a feedstuff comprising butyric acid and/or butyrate in accordance with the present invention, the microorganism can be added into the light corn steepwater at one time before conducting the fermentation. Alternatively, a part of microorganisms can be optionally added into the light corn steepwater before conducting the fermentation, and then the remained microorganisms can be added into the light corn steepwater at one time or in several batches during the fermentation. Optionally, additional light corn steepwater can be supplemented into the fermentation reactor during the fermentation. For example, the light corn steepwater can be mixed with the strain at one time before conducting the fermentation; or, the light corn steepwater can be divided into two or more batches of the same or different amounts, and then one batch is added into the reactor before conducting the fermentation, and the remained batches are separately added into the reactor during the fermentation.

Optionally, before conducting the method for preparing a feedstuff comprising butyric acid and/or butyrate in accordance with the present invention, the adopted first and/or second strain can be pre-cultured until they grow into the log phase. Such pre-cultured strains are used to perform the method of the present invention.

In the method for preparing a feedstuff comprising butyric acid and/or butyrate in accordance with the present invention, carbon sources, nitrogen sources and/or mineral elements could be optionally added into the mixture comprising light corn steepwater and microorganism before conducting the fermentation. Depending on the adopted first and second strain, the carbon source could be at least one of acetic acid, acetate and saccharides (e.g., glucose, sucrose and molasses). The mineral elements could be at least one of phosphorus, sulfur, potassium, magnesium, iron, and manganese, but are not limited thereto. For example, the mixture could be added with potassium dihydrogen phosphate (KH₂PO₄) to provide elements such as phosphorus and potassium, and could be added with magnesium chloride or magnesium sulfate heptahydrate (MgSO₄.7H₂O) to provide magnesium element, and/or could be added with ferric chloride or ferrous sulfate heptahydrate (FeSO₄.7H₂O) to provide an iron element.

After the fermentation is completed, the fermentation broth obtained from the method in accordance with the present invention can be used to manufacture animal feeds comprising butyric acid and/or butyrate. Depending on the desired uses of the feeds, the fermentation broth could be optionally subjected to a pH value modification and a condensation processing (e.g., evaporation-condensation). For example, sodium hydroxide, potassium hydroxide, calcium hydroxide, or calcium carbonate could be added into the fermentation broth to modify the pH value of the broth to be higher than 7. Alternatively, sulfuric acid, hydrochloric acid, formic acid, acetic acid, and/or lactic acid could be added into the fermentation broth to modify the pH value of the broth to be lower than 7.

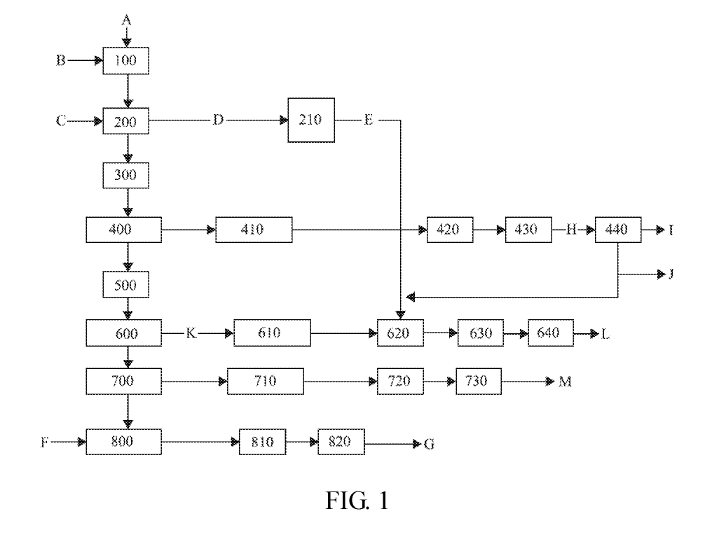

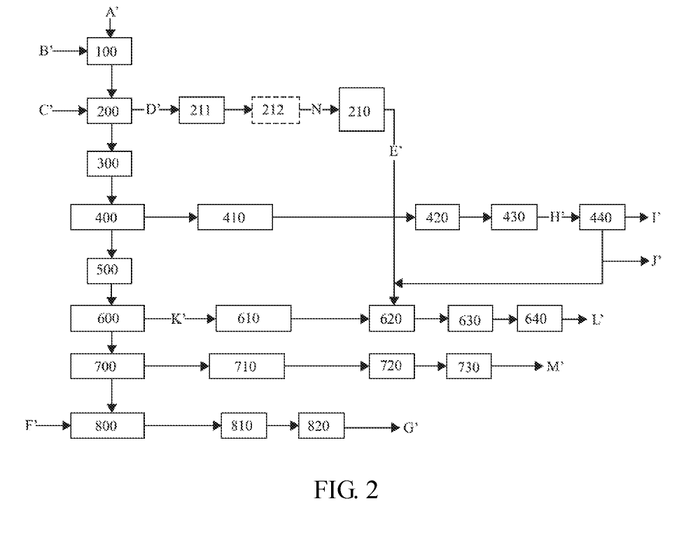

FIG. 2 is a diagram showing a processing flow for producing animal feeds from corn by applying the method in accordance with the present invention, wherein reference numerals identical to those in FIG. 1 represent the same processing or materials. As shown in FIG. 2, the method in accordance with the present invention can be applied in the processing flow of the typical wet-method for producing corn starch as shown in FIG. 1, wherein the light corn steepwater D′ obtained by steeping corn in the processing flow of wet-method is taken as the light corn steepwater of the method of the present invention. The light corn steepwater is subjected to fermentation 211 and optionally subjected to pH value modification 212 to provide butyric acid and/or butyrate-containing light corn steepwater N or condensed corn extractives E′. The light corn steepwater N or condensed corn extractives E′ can be used to produce a butyric acid and/or butyrate-containing corn gluten feed L′.

The present invention will be further illustrated in detail with specific examples as follows. However, the following examples are provided only for illustrating the present invention, and the scope of the present invention is not limited thereby. The scope of the present invention will be indicated in the appended claims.

### EXAMPLES

Sources or combinations of the materials used in the following examples are as follows:

### (a) Inorganic Components of CGM (Clostridial Growth Medium) Medium (pH 6.0):

Ammonium sulfate ((NH₄)₂SO₄): 3 g/L
Potassium dihydrogen phosphate (K₂HPO₄): 1.5 g/L
Magnesium sulfate heptahydrate (MgSO₄.7H₂O): 0.6 g/L
Ferrous sulfate heptahydrate (FeSO₄.7H₂O): 0.03 g/L

### (b) RCM (Reinforced Clostridial Medium) Medium (pH 6.8):

Meat extract: 10 g/L
Peptone: 10 g/L
Yeast extract: 3 g/L
D (+) glucose: 5 g/L
Starch: 1 g/L
NaCl: 5 g/L
Sodium acetate (CH3COONa): 3 g/L
L-cysteine chloride: 0.5 g/L
Agar: 0.5 g/L

In the following examples, an anaerobic atmosphere was provided in an air-tight container (e.g., air-tight bottle, fermentation bottle) by the following operations. The air-tight container and the rubber bung were covered with aluminum foil, and then sterilized under high temperature and high pressure (121° C., 1.2 atm) to exclude the interference of other microorganisms. Thereafter, the air-tight container was put in an oven to remove the residual moisture to prevent any microorganism contamination caused by the residual moisture. The dried air-tight container was transferred to an anaerobic operation box. After the sealing aluminum foil was slightly loosened, the palladium catalyst was used to catalyze the reaction of the oxygen in the air-tight container and the hydrogen in the anaerobic gas mixture to produce water and to deplete the oxygen in the air-tight container, and thus, provide an anaerobic atmosphere.

In the following examples, all the mediums were treated as follows to be deoxygenated. The prepared medium was sterilized under high temperature and high pressure (121° C., 1.2 atm) for 20 minutes, and then transferred into an anaerobic operation box before the medium cooled down to room temperature. Thereafter, the cap of the air-tight container in which the medium was kept was slightly loosened to release the steam contained therein. Then, with the use of the palladium catalyst, the reaction of the oxygen in the air-tight container and the hydrogen in the anaerobic gas mixture was catalyzed to produce water such that deoxygenation of the medium was performed. After the medium cooled down to room temperature, L-cysteine hydrochloride (0.5 g/L) was added thereinto to reduce the redox potential of the medium to a range suitable for microorganisms such that a deoxygenated medium was provided.

### Example 1: Preparation of Feedstuffs Comprising Butyric Acid and/or Butyrate

### 1-1. Selection of Strains

Clostridium tyrobutyricum DSM 2637, which is able to metabolize saccharides or organic compounds to produce organic acid (e.g., acetic acid and butyric acid) in fermentation, was used as the first strain. One of Terrisporobacter glycolicus DSM 1288 and Clostridium ljungdahlii DSM 13528, both are able to fix carbon oxide, was used as the second strain.

### 1-2. Pre-Culture

(a) Clostridium tyrobutyricum DSM 2637: a single colony of this strain was selected, inoculated in 10 ml deoxygenated RCM medium, and incubated in an anaerobic incubator at 37° C. for about 14 to 16 hours until the OD₆₀₀ (the absorbance at a wavelength of 600 nm) of the strain reached a value of about 1.0 to 1.2.
(b) Terrisporobacter glycolicus DSM 1288: a single colony of this strain was selected, inoculated in 10 ml deoxygenated RCM medium, and incubated in an anaerobic incubator at 37° C. for about 16 hours until the OD₆₀₀ (the absorbance at a wavelength of 600 nm) of the strain reached a value of about 1.0 to 1.2.
(c) Clostridium ljungdahlii DSM 13528: a single colony of this strain was selected, inoculated in 10 ml deoxygenated RCM medium that is externally added with 10 g/L fructose, and incubated in an anaerobic incubator at 37° C. for about 48 hours until the OD₆₀₀ (the absorbance at a wavelength of 600 nm) of the strain reached a value of about 1.0 to 1.2.

### 1-3. Fermentation Tests

### Test 1-3-1

A medium having a composition close to that of a light corn steepwater was prepared by mixing 200 ml of condensed corn extractives (purchased from Fonen and Fonher Enterprise Co., Ltd.) with 800 ml water. Sodium acetate (2 g) and inorganic components of CGM (i.e., 3 g of ammonium sulfate, 1.5 g of potassium dihydrogen phosphate, 0.6 g of magnesium sulfate heptahydrate, 0.03 g of ferrous sulfate heptahydrate) were added into the medium to provide a medium mixture. After the pH value of the medium mixture was adjusted to 6.5, 100 ml of this medium mixture was injected into an air-tight bottle, and then the medium mixture was deoxygenated.

Each of the pre-cultured Clostridium tyrobutyricum DSM 2637 and Terrisporobacter glycolicus DSM 1288 was inoculated into the above air-tight bottle at an inoculation rate of about 1%. The air-tight bottle was then kept in an anaerobic incubator at 37° C. to conduct fermentation, and the samples of 0-, 26- and 71-hour fermentation broth were collected. The samples were analyzed by an Agilent 1100 HPLC analysis in combination with an Aminex HPX-87H (300×7.8 mm) column to calculate the concentrations of acetic acid and butyric acid in the fermentation broth. The results are shown in Table 1. As shown in Table 1, the concentration of organic acid in fermentation broth could be controlled by the length of fermentation time.

TABLE 1

Composition of organic acid
in fermentation broth

| Sampling Time | Acetic acid | Butyric acid |
|---|---|---|
| (hours) | (g/L) | (g/L) |
| 0 | 3 | 0 |
| 26 | 0.59 | 7.53 |
| 71 | 0.36 | 9.41 |

### Test 1-3-2

The procedures of Test 1-3-1 were repeated with the exception that 1000 ml of acetic acid-containing light corn steepwater (purchased from Fonen And Fonher Enterprise Co., Ltd.) was used as the medium, so as to provide a medium mixture comprising 3 g of ammonium sulfate, 1.5 g of potassium dihydrogen phosphate, 0.6 g of magnesium sulfate heptahydrate, 0.03 g of ferrous sulfate heptahydrate and 8.6 g of acetic acid per liter. After the pH value of the medium mixture was adjusted to 6.3, 100 ml of this medium mixture was injected into an air-tight bottle, and then the medium mixture was deoxygenated.

Each of the pre-cultured Clostridium tyrobutyricum DSM 2637 and Terrisporobacter glycolicus DSM 1288 was inoculated into the above air-tight bottle at an inoculation rate of about 1.5%. The air-tight bottle was then kept in an anaerobic incubator at 37° C. to conduct fermentation and the samples of 0- and 104-hours fermentation broth were collected. The samples were analyzed by an Agilent 1100 HPLC analysis in combination with an Aminex HPX-87H (300×7.8 mm) column to calculate the concentrations of acetic acid and butyric acid in the fermentation broth. The results are shown in Table 2. As shown in Table 2, the concentration of organic acid in fermentation broth could be controlled by the length of fermentation time.

TABLE 2

Composition of organic acid
in fermentation broth

| Sampling Time | Acetic acid | Butyric acid |
|---|---|---|
| (hours) | (g/L) | (g/L) |
| 0 | 8.6 | 0 |
| 104 | 0.11 | 20.96 |

### Test 1-3-3

A medium having a composition close to that of a light corn steepwater was prepared by mixing 100 g of corn steep powder (CSP; purchased from Roquette freres company, product name: Solulys 095E) with water (to a total volume of 1000 ml). Sodium acetate (5 g) and inorganic components of CGM (i.e., 3 g of ammonium sulfate, 1.5 g of potassium dihydrogen phosphate, 0.6 g of magnesium sulfate heptahydrate, 0.03 g of ferrous sulfate heptahydrate) were added into the medium to provide a medium mixture. After the pH value of the medium mixture was adjusted to 6.4, 100 ml of this medium was injected into an air-tight bottle, and then the medium mixture was deoxygenated.

The pre-cultured Clostridium tyrobutyricum DSM 2637 and Clostridium ljungdahlii DSM 13528 were both inoculated into the above air-tight bottle at an inoculation rate of about 2% and 5%, respectively. The air-tight bottle was then kept in an anaerobic incubator at 37° C. and the samples of 0-, 24- and 72-hour fermentation broth were collected. The samples were analyzed by an Agilent 1100 HPLC analysis in combination with an Aminex HPX-87H (300×7.8 mm) column to calculate the concentrations of acetic acid and butyric acid in the fermentation broth. The results are shown in Table 3. As shown in Table 3, the concentration of organic acid in fermentation broth could be controlled by the length of fermentation time.

TABLE 3

Composition of organic acid
in fermentation broth

| Sampling Time | Acetic acid | Butyric acid |
|---|---|---|
| (hours) | (g/L) | (g/L) |
| 0 | 4.3 | 0 |
| 24 | 0 | 12.8 |
| 72 | 0 | 13.39 |

### Test 1-3-4

A medium having a composition close to that of a light corn steepwater was prepared by mixing 300 g of corn steep powder (CSP; purchased from Roquette freres company, product name: Solulys 095E) with water (to a total volume of 2700 ml). Sodium acetate (15 g) and the inorganic components of CGM (e.g., 9 g of ammonium sulfate, 4.5 g of potassium dihydrogen phosphate, 1.8 g of magnesium sulfate heptahydrate, 0.09 g of ferrous sulfate heptahydrate) were added into the medium. Then, the medium was adjusted to a pH value of 6.0, and then deoxygenated. The medium mixture thus obtained was used for performing the first, second and third batch fermentations.

First batch fermentation: 900 ml of the above mixture medium was injected into a stirring fermentation bottle. Each of the pre-cultured Clostridium tyrobutyricum DSM 2637 and Terrisporobacter glycolicus DSM 1288 was inoculated at an inoculation rate of about 10%. The stirring fermentation bottle was kept in a water bath at 37° C. The pH value of the medium therein was controlled at 6.0. The samples of 0-, 15.5- and 22.5-hours fermentation broth were collected. The samples were analyzed by an Agilent 1100 HPLC analysis in combination with an Aminex HPX-87H (300×7.8 mm) column to calculate the concentrations of acetic acid and butyric acid in the fermentation broth. The results are shown in Table 4. As shown in Table 4, the concentration of organic acid in fermentation broth could be controlled by the length of fermentation time.

Second batch of fermentation: The fermentation broth of the first batch was removed and only 10% of the fermentation broth was retained (i.e., 10% inoculation rate). Thereafter, 900 ml of the above medium mixture was injected into the fermentation bottle and kept in a water bath at 37° C. The pH value of the medium therein was controlled at 6.0. The samples of 0-, 16.5- and 25-hours fermentation broth were collected. The samples were analyzed by an Agilent 1100 HPLC analysis in combination with an Aminex HPX-87H (300×7.8 mm) column to calculate the concentrations of acetic acid and butyric acid in the fermentation broth of the samples. The results are shown in Table 4.

Third batch of fermentation: The fermentation broth of the second batch was removed and only 10% of the fermentation broth was retained (i.e., 10% inoculation rate). Thereafter, 900 ml of the above medium mixture was injected into the fermentation bottle and kept in a water bath at 37° C. The pH value of the medium therein was controlled at 6.0. The samples of 0- and 48-hours fermentation broth were collected. The samples were analyzed by an Agilent 1100 HPLC analysis in combination with an Aminex HPX-87H (300×7.8 mm) column to calculate the concentrations of acetic acid and butyric acid in the fermentation broth. The results are shown in Table 4.

TABLE 4

Composition of organic acid
in fermentation broth

| Batch of | Sampling Time | Acetic acid | Butyric acid |
|---|---|---|---|
| fermentation | (hours) | (g/L) | (g/L) |
| 1^{st} | 0 | 4.3 | 0 |
| | 15.5 | 0 | 11.6 |
| | 22.5 | 0 | 12.0 |
| 2^{nd} | 0 | 3.83 | 1.87 |
| | 16.5 | 0 | 11.4 |
| | 25 | 0 | 12.3 |
| 3^{rd} | 0 | 4.1 | 1.5 |
| | 48 | 2.1 | 13.3 |

### Test 1-3-5

A medium having a composition close to that of a light corn steepwater was prepared by mixing 100 g of corn steep powder (CSP; purchased from Roquette freres company, product name: Solulys 095E) with water (to a total volume of 900 ml). Sodium acetate (5 g) and inorganic components of CGM (i.e., 3 g of ammonium sulfate, 1.5 g of potassium dihydrogen phosphate, 0.6 g of magnesium sulfate heptahydrate, 0.03 g of ferrous sulfate heptahydrate) were added into the medium to provide a medium mixture. After the pH value of the medium mixture was adjusted to 5.8, this medium was injected into a stirring fermentation bottle, and then the medium mixture was deoxygenated.

In the above stirring fermentation bottle, each of the pre-cultured Clostridium tyrobutyricum DSM 2637 and Terrisporobacter glycolicus DSM 1288 was inoculated at an inoculation rate of about 10%. Thereafter, the fermentation bottle was kept in a water bath at 37° C. The samples of 0-, 21.5- and 40.5-hours were collected. The samples were analyzed by an Agilent 1100 HPLC analysis in combination with an Aminex HPX-87H (300×7.8 mm) column to calculate the concentrations of glucose, acetic acid, crude protein and butyric acid in the fermentation broth. During the fermentation, the pH value was not controlled, and the pH value of the 40.5-hour fermentation broth was 7.24. The results are shown in Table 5. The concentration of crude protein was obtained by multiplying the total nitrogen content obtained from Simplified TKN (s-TKN™) analysis by 6.25.

TABLE 5

| | Composition of fermentation broth | | | |
|---|---|---|---|---|
| Sampling Time | Glucose | Acetic acid | Crude protein | Butyric acid |
| (hours) | (g/L) | (g/L) | (g/L) | (g/L) |
| 0 | 0.12 | 4.42 | 60.19 | 0.11 |
| 21.5 | 0 | 1.26 | — | 12.18 |
| 40.5 | 0 | 4.11 | 63.01 | 14.23 |

### Test 1-3-6

A medium having a composition close to that of a light corn steepwater was prepared by mixing 100 g of corn steep powder (CSP; purchased from Roquette freres company, product name: Solulys 095E) with water (to a total volume of 900 ml). Sodium acetate (5 g) and inorganic components of CGM (i.e., 3 g of ammonium sulfate, 1.5 g of potassium dihydrogen phosphate, 0.6 g of magnesium sulfate heptahydrate, 0.03 g of ferrous sulfate heptahydrate) were added into the medium to provide a medium mixture. After the pH value of the medium mixture was adjusted to 5.8, this medium was injected into a stirring fermentation bottle, and then the medium mixture was deoxygenated.

In the above stirring fermentation bottle, the pre-cultured Clostridium tyrobutyricum DSM 2637 was inoculated at an inoculation rate of about 10%. Thereafter, the fermentation bottle was kept in a water bath at 37° C. The samples of 0-, 22- and 40.5-hours fermentation broth were collected. The samples were analyzed by an Agilent 1100 HPLC analysis in combination with an Aminex HPX-87H (300×7.8 mm) column to calculate the concentrations of glucose, acetic acid, crude protein and butyric acid in the fermentation broth. During fermentation, the pH value was not controlled. After the fermentation, the pH value of the fermentation broth was 7.6. The results are shown in Table 6. The concentration of crude protein was obtained by multiplying the total nitrogen content obtained from Simplified TKN (s-TKN™) analysis by 6.25.

TABLE 6

| | Composition of fermentation broth | | | |
|---|---|---|---|---|
| Sampling Time | Glucose | Acetic acid | Crude protein | Butyric acid |
| (hours) | (g/L) | (g/L) | (g/L) | (g/L) |
| 0 | 0 | 6.33 | 69.8 | 0.16 |
| 22 | 0 | 1.34 | — | 16.04 |
| 40.5 | 0 | 1.21 | 72.8 | 15.82 |

### Test 1-3-7

A medium having a composition close to that of a light corn steepwater was prepared by mixing 100 g of corn steep powder (CSP; purchased from Roquette freres company, product name: Solulys 095E) with water (to a total volume of 900 ml). Sodium acetate (5 g) and inorganic components of CGM (i.e., 3 g of ammonium sulfate, 1.5 g of potassium dihydrogen phosphate, 0.6 g of magnesium sulfate heptahydrate, 0.03 g of ferrous sulfate heptahydrate) were added into the medium to provide a medium mixture. After the pH value of the medium mixture was adjusted to 5.5, 50 ml of the medium mixture was injected into an air-tight bottle, and then the medium mixture was deoxygenated.

In the above fermentation bottle, the pre-cultured Clostridium tyrobutyricum DSM 2637 was inoculated at an inoculation rate of about 10%. Thereafter, the fermentation bottle was kept in a water bath at 37° C. The samples of 0-, 1-, 2-, 3-, 4- and 5-hours fermentation broth were collected. The samples were analyzed by an Agilent 1100 HPLC analysis in combination with an Aminex HPX-87H (300×7.8 mm) column to calculate the concentrations of acetic acid, propionic acid and butyric acid in the fermentation broth (i.e., steepwater comprising butyric acid and/or butyrate). During the fermentation, the pH value was not controlled. The results are shown in Table 7. As shown in Table 7, the concentration of organic acid in fermentation broth could be controlled by the length of fermentation time.

In Table 7, the butyric acid content of fermentation broth that was detected in the 0-hour fermentation broth was not originally present in the light corn steepwater, but brought from the inoculation of the pre-cultured Clostridium tyrobutyricum DSM 2637 at an inoculation rate of about 10%.

TABLE 7

The composition of organic acid
in fermentation broth

| Sampling Time | Acetic acid | Propionic acid | Butyric acid |
|---|---|---|---|
| (hours) | (g/L) | (g/L) | (g/L) |
| 0 | 0.9 | 0.18 | 0.21 |
| 1 | 0.83 | 0.18 | 0.42 |
| 2 | 0.74 | 0.17 | 0.68 |
| 3 | 0.59 | 0.17 | 1.04 |
| 4 | 0.47 | 0.18 | 1.31 |
| 5 | 0.36 | 0.2 | 1.59 |

As shown in the results of the above Test 1-3-1 to Test 1-3-7, in the processing flow for manufacturing feeds from corn by applying the method in accordance with the present invention, the first strain or both the first and second strain were added into the obtained light corn steepwater. After the fermentation was conducted, the light corn steepwater comprising butyric acid and/or butyrate that met the requirement of fodder-grade could be directly provided without changing the order of the traditional processing flow. The light corn steepwater comprising butyric acid and/or butyrate could be used for preparing animal feeds.

### Example 2: Preparation of Condensed Corn Extractives Comprising Butyric Acid and/or Butyrate

### Test 2-1

706 g of the 40.5-hour fermentation broth provided by Test 1-3-5 (i.e., a butyric acid and/or butyrate-containing light corn steepwater) was injected into a one-liter round-bottomed flask with a stirrer, and heated at a stress of −660 mmHg to evaporate moisture. The product with a solid content of about 50 wt. % thus obtained was a butyric acid and/or butyrate-containing condensed corn extractives. After cooling down, the product (i.e., the condensed corn extractives) was analyzed by Agilent 1100 HPLC analysis in combination with Aminex HPX-87H (300×7.8 mm) column to calculate the concentrations of glucose, acetic acid, crude protein and butyric acid therein. The results are shown in Table 8.

TABLE 8

| Glucose | Acetic acid | Crude protein | Butyric acid |
|---|---|---|---|
| (g/L) | (g/L) | (g/L) | (g/L) |
| 0 | 20.7 | 281.5 | 71.9 |

### Test 2-2

701 g of the 40.5-hour fermentation broth provided by Test 1-3-6 (i.e., a butyric acid and/or butyrate-containing light corn steepwater) was injected into a one-liter round-bottomed flask with a stirrer and heated at a stress of −660 mmHg to evaporate moisture. The product with a solid content of about 50 wt. % thus obtained was a butyric acid and/or butyrate-containing condensed corn extractives. After cooling down, the product (i.e., the condensed corn extractives) was analyzed by an Agilent 1100 HPLC analysis in combination with an Aminex HPX-87H (300×7.8 mm) column to calculate the concentration of glucose, acetic acid, crude protein and butyric acid therein. The results are shown in Table 9.

TABLE 9

| Glucose | Acetic acid | Crude protein | Butyric acid |
|---|---|---|---|
| (g/L) | (g/L) | (g/L) | (g/L) |
| 0 | 3.5 | 307.8 | 71.65 |

As shown in the results of Test 2-1 and 2-2, the butyric acid and/or butyrate-containing light corn steepwater provided by Test 1-3-5 or 1-3-6 was subjected to an evaporation-condensation, and the condensed corn extractives thus obtained was of a solid content of about 50 wt. % and a fivefold increased concentration of butyric acid. Therefore, if the butyric acid and/or butyrate-containing light corn steepwater provided by Test 1-3-7 was evaporation-condensed by the same method of Test 2-1 and 2-2, the concentration of butyric acid in the condensed corn extractives thus obtained (solid content is about 50 wt. %) would be the value as shown in Table 10.

TABLE 10

| Sampling | Estimated value of the content of butyric |
|---|---|
| Time | acid in condensed corn extractives |
| (hours) | (g/L) |
| 0 | 1 |
| 1 | 2.1 |
| 2 | 3.4 |
| 3 | 5.2 |
| 4 | 6.5 |
| 5 | 8.0 |

### BRIEF DESCRIPTION OF REFERENCE NUMERALS

A, A′: corn kernel
B, B′: water
C, C′: sulfur dioxide
D, D′: light corn steepwater
E: condensed corn extractives
E′: butyric acid and/or butyrate-containing condensed corn extractives
F, F′: water
G′: corn starch
H, H′: germ
I, I′: crude corn oil
J, J′: corn germ meal
K, K′: corn hull fiber
L: corn gluten feed
L′: butyric acid and/or butyrate-containing corn gluten feed
M, M′: gluten powder
N: butyric acid and/or butyrate-containing light corn steepwater
100: cleansing processing
200: steeping processing
210: evaporation-condensation
211: fermentation
212: pH value modification
300: breakdown processing
400: germ isolation processing
410: germ wash
420: dehydration
430: drying
440: oil press processing
500: fine grinding processing
600: fiber isolation processing
610: wash and dehydration
620: mixing
630: drying
640: granulation
700: protein isolation processing
710: evaporation-condensation
720: dehydration
730: drying
800: starch wash
810: dehydration
820: drying

### Deposit of Biological Material

### Deposit Information:

1. Clostridium tyrobutyricum: DE, Germany, Deutsche Sammlung von Mikroorganismen und Zellkulturen GmbH, DSM 2637.
2. Terrisporobacter glycolicus: DE, Germany, Deutsche Sammlung von Mikroorganismen und Zellkulturen GmbH, DSM 1288.
3. Clostridium ljungdahlii: DE, Germany, Deutsche Sammlung von Mikroorganismen und Zellkulturen GmbH, DSM 13528.

What is claimed is:

1. A method for preparing a feedstuff comprising butyric acid and/or butyrate, comprising:
adding a microorganism into a light corn steepwater to provide a mixture, wherein the microorganism comprises a first strain and the first strain is able to metabolize saccharides and/or organic compounds in a fermentation to produce butyric acid;
keeping the mixture under an anaerobic atmosphere to conduct the fermentation to provide a fermentation broth; and
optionally condensing the fermentation broth.

2. The method as claimed in claim 1, wherein the first strain is at least one of Clostridium sp. strain, Butyribacterium sp. strain, and Butyrivibrio sp. strain.

3. The method as claimed in claim 1, wherein the first strain is at least one of Clostridium tyrobutyricum, Clostridium butyricum, Clostridium beijerinckii, Clostridium acetobutylicum, Clostridium argentinense, Clostridium aurantibutyricum, Clostridium botulinum, Clostridium carboxidivorans, Clostridium cellulovorans, Clostridium cf. saccharolyticum, Clostridium difficile, Clostridium kluyveri, Clostridium novyi, Clostridium paraputrificum, Clostridium pascui, Clostridium pasteurianum, Clostridium peptidivorans, Clostridium perfringens, Clostridium scatologenes, Clostridium schirmacherense, Clostridium sticklandii, Clostridium subterminale SB4, Clostridium symbiosum, Clostridium tetani, Clostridium tepidiprofundi, Clostridium tertium, Clostridium tetanomorphum, and Clostridium thermopalmarium.

4. The method as claimed in claim 1, wherein the light corn steepwater comprises crude protein, ash, carbohydrate, and fat.

5. The method as claimed in claim 1, wherein the first strain is Clostridium tyrobutyricum, and the fermentation is conducted at a temperature of 32 to 42° C.

6. The method as claimed in claim 1, wherein the method further comprises adding carbon sources, nitrogen sources and/or mineral elements into the mixture before conducting the fermentation.

7. The method as claimed in claim 6, wherein the carbon source is at least one of acetic acid, acetate and saccharide, and the mineral element is at least one of phosphorus, sulfur, potassium, magnesium, iron, and manganese.

8. The method as claimed in claim 1, wherein the microorganism further comprises a second strain, and the second strain is able to fix a carbon oxide.

9. The method as claimed in claim 8, wherein the second strain uses the Wood Ljungdahl (WL) pathway to fix a carbon oxide.

10. The method as claimed in claim 9, wherein the second strain is at least one of Clostridium coskatii, Clostridium ljungdahlii, Clostridium autoethanogenum, Clostridium ragsdalei, Terrisporobacter glycolicus, and Clostridium scatologenes.

11. The method as claimed in claim 8, wherein the first strain is at least one of Clostridium sp. strain, Butyribacterium sp. strain, and Butyrivibrio sp. strain.

12. The method as claimed in claim 8, wherein the first strain is at least one of Clostridium tyrobutyricum, Clostridium butyricum, Clostridium beijerinckii, Clostridium acetobutylicum, Clostridium argentinense, Clostridium aurantibutyricum, Clostridium botulinum, Clostridium carboxidivorans, Clostridium cellulovorans, Clostridium cf. saccharolyticum, Clostridium difficile, Clostridium kluyveri, Clostridium novyi, Clostridium paraputrificum, Clostridium pascui, Clostridium pasteurianum, Clostridium peptidivorans, Clostridium perfringens, Clostridium scatologenes, Clostridium schirmacherense, Clostridium sticklandii, Clostridium subterminale SB4, Clostridium symbiosum, Clostridium tetani, Clostridium tepidiprofundi, Clostridium tertium, Clostridium tetanomorphum, and Clostridium thermopalmarium.

13. The method as claimed in claim 8, wherein the light corn steepwater comprises crude protein, ash, carbohydrate, and fat.

14. The method as claimed in claim 8, wherein the method further comprises adding carbon sources, nitrogen sources and/or mineral elements into the mixture before conducting the fermentation.

15. The method as claimed in claim 14, wherein the carbon source is at least one of acetic acid, acetate and saccharide, and the mineral element is at least one of phosphorus, sulfur, potassium, magnesium, iron, and manganese.
